(19) United States
(12) Patent Application Publication
(10) Pub. No.: US 2018/0263259 A1
(43) Pub. Date: Sep. 20, 2018

(54) METHODS AND USES FOR KERATIN HYDROLYSIS

(71) Applicant: DuPont Nutrition Biosciences ApS, Copenhagen K (DK)

(72) Inventors: Jon Gade, Aarhus V (DK); KARSTEN MATTHIAS KRAGH, Hoejbjerg (DK); Stepan Shipovskov, Ega (DK); Shukun Yu, Bunkeflostrand (SE)

(21) Appl. No.: 15/542,292

(22) PCT Filed: Jan. 7, 2016
(86) PCT No.: PCT/EP2016/050173

§ 371 Date: Jul. 7, 2017

Related U.S. Application Data
(60) Provisional application No. 62/100,811, filed on Jan. 7, 2015.
(60) Provisional application No. 62/164,961, filed on May 21, 2015.

(51) Int. Cl. A23J 3/34; C12P 21/06

(52) U.S. Cl. CPC A23J 3/341; C07K 14/4741; C12P 21/06

(57) ABSTRACT

The present disclosure provides methods and uses for keratin hydrolysates (e.g. feathers hydrolysates).

### BACKGROUND

Keratin is the collective name for a family of though proteins which are found in a number of structures. The degradation of keratin can be of significant commercial value. One source of keratin in particular—feathers—is produced in vast quantities by the poultry industry. In 2002 approximately 49 billion chickens were utilized in the poultry industry. Poultry feathers typically contain approximately 90% protein in the form of 13-keratin. However, keratin must be cleaved before its protein content can be digested by animals. Degradation of feathers can therefore provide an inexpensive source of digestible protein and amino acids. Accordingly feather hydrolysate (i.e. degraded feathers) can be utilized in a numbers of ways, such as in animal feed. However, current methods of recovering this nutriment are so inefficient and costly that the vast majority of keratin waste streams are simply disposed of in landfill or via incineration, both of which can cause environmental problems and reduce the sustainability of the main commercial process (often meat production for human consumption).

Unfortunately some contemporary processes for the production of keratin hydrolysate produce feather meal that is more expensive than chicken meat. In addition, some of the processes produce incomplete hydrolysis, contamination with undesirable amino acids, and, some methods have not been successful in producing highly digestible products. The present disclosure solves those problems by providing methods, apparatuses, compositions and kits that are economically viable for the enzymatic hydrolysis of keratin (e.g. in feathers) compared to conventional methods and produce digestible and more palatable products.

### SUMMARY OF THE INVENTION

The present disclosure provides methods, apparatuses, compositions and kits for the enzymatic hydrolysis of keratin (e.g. in feathers).

Aspects and embodiments of the compositions and methods are set forth in the following separately numbered paragraphs.

1. A method of production of keratin hydrolysate comprising the steps of:
a) processing a keratin-containing material in a size-reduction process to produce a size-reduced keratin material, where the size-reduced keratin material comprises an average particle size of 5 mm or less,
b) admixing the size-reduced keratin material with one or more proteases, and
c) hydrolyzing the keratin material by heat hydrolysis, where step b) and c) can occur in any order.

2. The method of paragraph 2, where the heat hydrolysis is steam hydrolysis.

3. The method of paragraph 2, where the steam has a temperature of from about 120° C. to about 140° C.

4. The method of any preceding paragraph, where the heat hydrolysis occurs prior to the enzyme hydrolysis step.

5. The method of any preceding paragraph, where the steam or heat hydrolysis occurs simultaneously with the size-reducing step prior to the enzyme hydrolysis step.

6. The method of paragraph 1 or 2, where the heat hydrolysis occurs after to the enzyme hydrolysis step.

7. The method of any preceding paragraph, where no further chemical hydrolysis of the keratin material is performed.

8. The method of any preceding paragraph, where the protease is a subtilisin, a serine protease, a metalloprotease, an acid protease, or a neutral protease.

9. The method of paragraph 8, where the protease comprises an amino acid sequence which has at least 80, 81, 82, 83, 84, 85, 86, 87, 88, 89, 90, 91, 92, 93, 94, 95, 96, 97, 98 or 99% identity with the amino acid SEQ ID No.1.

10. The method of paragraph 9, where the protease comprises an amino acid sequence which has at least 90% identity with the amino acid SEQ ID No.1.

11. The method of paragraph 10, where the protease comprises an amino acid sequence which has at least 95% identity with the amino acid SEQ ID No.1.

12. The method of paragraph 8, where the protease comprises an amino acid sequence which has at least 80, 81, 82, 83, 84, 85, 86, 87, 88, 89, 90, 91, 92, 93, 94, 95, 96, 97, 98 or 99% identity with the amino acid SEQ ID No.2.

13. The method of paragraph 12, where the protease comprises an amino acid sequence which has at least 90% identity with the amino acid SEQ ID No.2.

14. The method of paragraph 13, where the protease comprises an amino acid sequence which has at least 95% identity with the amino acid SEQ ID No.2.

15. The method of any preceding paragraph, where the heat hydrolysis step is carried out at +/− about 1 pH of the optimal pH of the protease.

16. The method of any preceding paragraph, where the heat hydrolysis step is carried out at +/− about 0.5 pH of the optimal pH of the protease.

17. The method of any preceding paragraph, where the optimal pH of the protease is 6-10.

18. The method of any preceding paragraph, where the keratin material is feathers.

19. The method of any preceding paragraph, where the keratin material is reduced to an average particle size of about 4.5 mm or less, or less than 4 mm, or less than 3 mm, or less than 2 mm, or less than 1 mm.

20. The method of any preceding paragraph where the protease is in a liquid composition.

21. The method of paragraph 20 where the liquid composition comprise protease activity of 2500-3600 U/g.

22. The method of any preceding paragraph where the protease is present in an amount of 0.3 g-9 g per Kg of keratin-containing proteinaceous material.

23. The method of paragraph 22, where the protease comprise protease activity of 2500-3600 U/g.

24. The method of any preceding paragraph, where the method is carried out in an oxygen controlled environment.

25. The method of any preceding paragraph, where the method is carried out as a batch, fed-batch or continuous process.

26. A keratin hydrolysate comprising at least about 50% of peptides with N-terminal Leucine, Isoleucine, Valine, Alanine, Tyrosine, Phenylalanine or Lysine.

27. The keratin hydrolysate of paragraph 26 comprising at least about 70% of peptides with N-terminal Leucine, Isoleucine, Valine, Alanine, Tyrosine, Phenylalanine or Lysine.

28. The keratin hydrolysate of paragraph 26 comprising at least about 80% of peptides with N-terminal Leucine, Isoleucine, Valine, Alanine, Tyrosine, Phenylalanine or Lysine.

29. The keratin hydrolysate of paragraph 26 comprising at least about 90% of peptides with N-terminal Leucine, Isoleucine, Valine, Alanine, Tyrosine, Phenylalanine or Lysine.

30. The keratin hydrolysate of paragraphs 26-29, where 80% of the keratin hydrolysates comprise peptides having a molecular weight in the range of from about 0.1 kDa to about 5 kDa.

31. The keratin hydrolysate of paragraphs 26-30, where 80% of the keratin hydrolysates comprise peptides having a molecular weight in the range of from about 0.1 kDa to about 1.0 kDa.

32. The keratin hydrolysate of paragraphs 26-31, where 80% of the keratin hydrolysates comprise peptides having a molecular weight in the range of from about 0.1 kDa to about 0.8 kDa.

33. The keratin hydrolysate of paragraphs 26-32, where the keratin hydrolysates have 50% less undesirable amino acids that result from chemical hydrolysis when compared to a comparable keratin hydrolysate produced using chemical hydrolysis.

### BRIEF DESCRIPTION OF THE DRAWINGS

A better understanding of the features and advantages of the present invention will be obtained by reference to the following detailed description that sets forth illustrative embodiments, in which the principles of the invention are utilized, and the accompanying drawings of which:

FIG. 1 depicts FAO Food Outlook November 2012: Review by Fish Product.

FIG. 2 depicts chicken feathers as an enzymatic substrate.

FIG. 3 shows an enzymatic feather hydrolysis process.

FIG. 4 shows a method for evaluating pepsin-HCl digestibility.

FIG. 5 shows an embodiment for the use of enzymatic feather hydrolysis.

FIG. 6 shows results for feather hydrolysis replacement.

FIG. 7 shows an embodiment for the use of enzymatic feather hydrolysis.

FIG. 8 shows results for feather hydrolysis with protease pretreatment.

FIG. 9 shows an embodiment for the use of enzymatic feather hydrolysis.

FIG. 10 shows results for feather hydrolysis for protease post treatment.

FIGS. 11 A and B show Seq ID No. 1 and Seq ID. No. 2, respectively.

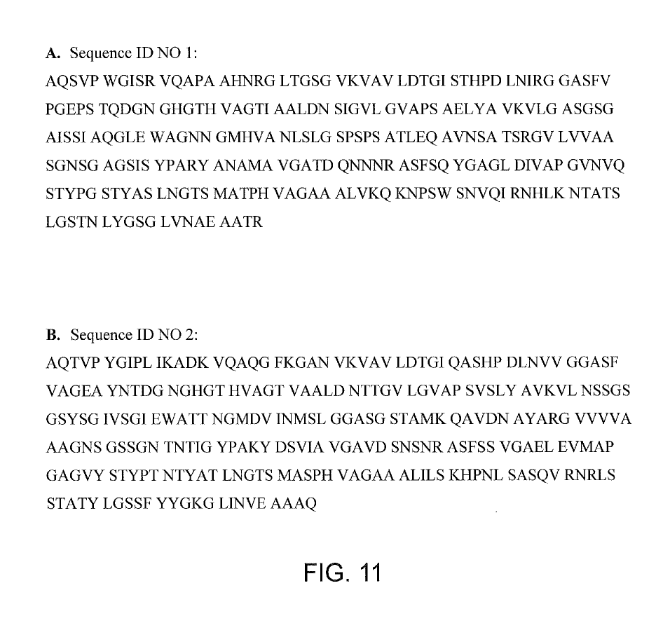

### DETAILED DESCRIPTION OF THE INVENTION

The present disclosure provides methods, apparatuses, compositions and kits that are economically viable for the enzymatic hydrolysis of keratin (e.g. in feathers) compared to conventional methods. In some embodiments, the present disclosure provides methods, apparatuses, compositions and kits for the use of feather meal as a new source of protein for pet food.

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide one of skill with a general dictionary of many of the terms used in this disclosure.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

The headings provided herein are not limitations of the various aspects or embodiments of this disclosure which can be had by reference to the specification as a whole. Accordingly, the terms defined immediately below are more fully defined by reference to the specification as a whole.

It must be noted that as used herein and in the appended claims, the singular forms “a”, “an”, and “the” include plural referents unless the context clearly dictates otherwise. Thus, for example, reference to “a protease” includes a plurality of such enzymes and reference to “the feed” includes reference to one or more feeds and equivalents thereof known to those skilled in the art, and so forth.

The publications discussed herein are provided solely for their disclosure prior to the filing date of the present application. Nothing herein is to be construed as an admission that such publications constitute prior art to the claims appended hereto.

### Keratin Materials

Keratin molecules are helical and fibrous, twisting around each other to form strands called intermediate filaments. It is thought that this makes a lot of the protein inaccessible to enzyme digestion at first instance. Additionally, keratin proteins contain a high percentage of sulfur-containing amino acids, largely cysteine, which form disulfide bridges between the individual molecules and assist in forming the fairly rigid structure of keratin. Unfortunately, the disulphide bridges also make digestion and degradation of the keratin rather difficult. There are two main types of keratin, alpha- and beta-keratins. The α-keratins are mostly present in the hair (including wool), horns, nails, claws and hooves of mammals. The harder β-keratins are found in nails and in the scales and claws of reptiles, their shells (Testudines, such as tortoise, turtle, terrapin), and in the feathers, beaks, claws of birds and quills of porcupines. β-keratins are formed primarily in beta sheets, although some beta sheets are also found in α-keratins.

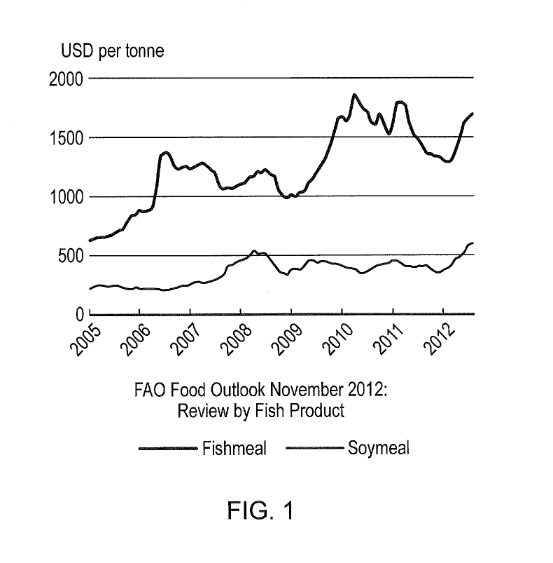

One source of keratin in particular—feathers—is produced in vast quantities by the poultry industry. Feather is a protein rich by-product from the poultry industry with approximately 80 million tons chickens worldwide. It is approximately 5-7 w/w % of total chicken, approximately 4.5 million tons feather protein worldwide. FIG. 1 depicts FAO Food Outlook November 2012: Review by Fish Product.

Poultry feathers typically contain approximately 90% protein in the form of β-keratin. However, keratin must be cleaved before its protein content can be digested by animals (McCasland and Richardson 1966, Poult. Sci., 45:1231-1236; Moran et al. 1966 Poult. Sci., 45: 1257-1266). Degradation of feathers can therefore provide an inexpensive source of digestible protein and amino acids. Accordingly feather hydrolyzate (i.e. degraded feathers) can be utilized in a numbers of ways, such as in animal feed. However, current methods of recovering this nutriment are so inefficient and costly that the vast majority of keratin waste streams are simply disposed of in landfill or via incineration, both of which can cause environmental problems and reduce the sustainability of the main commercial process (often meat production for human consumption). Unfortunately some contemporary processes for the production of keratin hydrolyzate produce feather meal that is more expensive than chicken meat.

High temperature and pressure is used to produce conventional feather meal for feed, pet food. Keratin-containing materials can be denatured by subjecting them to harsh physical conditions such as relatively high heat and pressure, for example at 146° C. and 345 kPa for about 30 to 70 minutes. Such treatment can facilitate breakdown of the sulfide bonds, but only incompletely hydrolyzes the keratin. In addition, such conditions are destructive to certain amino acids and may lead to the production of undesirable sulfur-containing non-nutritive amino acids in the end products.

Chemical treatment may also be used to break the disulfide bonds and can generate relatively shorter peptides from keratin. For example, boiling keratin for about 2 to 20 hours at a pH less than or equal to 2.0 to 4.0, or boiling for more than two hours at a highly alkaline pH yielded oligopeptides, polypeptides and free amino acids. However, such harsh treatments may partially or completely destroy certain amino acids, thereby reducing the nutritional aspects of the end product. Alkaline hydrolysis in particular tends to yield undesirable artificial amino acids such as lanthionine and lysinoalanine, the latter of which has been implicated as a renal toxic factor in laboratory rats. Treatment with acidic or basic materials may also produce residual salts in the mixture, which may necessitate additional processing steps for removal.

Thus, hydrolysis of keratin-containing materials by means of harsh treatments such as heat and chemicals suffers from the problems of incomplete hydrolysis and contamination of the food product with undesirable amino acids and residual salts. Moreover, such methods have not been successful in producing highly digestible food products. These methods generally do not yield products having digestibility greater than about 80% as measured by the 2-step method of Boisen and Fernandez (1995). Consequently, conventional methods suffer from low digestibility, high energy costs and risks in worker safety (H₂S production).

The present invention provides methods, apparatuses, compositions and kits for converting keratin-containing proteinaceous material into a desirable food product ingredient that is nutritious, palatable, and highly digestible by an animal. The present invention provides methods, apparatuses, compositions and kits for use to pretreat protein-containing raw materials, particularly, abundant, sustainable, low cost keratin-containing raw materials, under mild conditions to loosen tightly-packed beta-sheet structure and to efficiently break the peptide bonds of the keratin. The present invention provides methods, apparatuses, compositions and kits for the production of food products relatively free of undesirable amino acids, and increase palatability and digestibility.

Without intending to be limited to any theory, the present inventors have surprisingly found an improved process providing steps to loosen tightly-packed beta-sheet structure, and to allow enzymatic hydrolysis to efficiently break the peptide bonds of the keratin. In some embodiments, the invention provides a process that does not need the use of chemicals for chemical hydrolysis of the peptides. The present invention provides an improved process for preparing food protein ingredients that involves a first step of reducing the size of the keratin-containing material (e.g. raw materials such as feathers) to a desire average particle size. The sized-reduced keratin material is then subjected to enzyme hydrolysis by mixing a quantity of at least one proteolytic enzyme with the pretreated sized-reduced keratin material. In some embodiments, the process also involves steam or heat hydrolysis prior or after the enzyme hydrolysis. In some embodiments, the steam or heat hydrolysis occurs prior to the enzyme hydrolysis step. In some embodiments, the steam or heat hydrolysis occurs simultaneously with the size-reducing step prior to the enzyme hydrolysis step. The protein hydrolysate may be then further subjected to further processes to produce a food or feed ingredient.

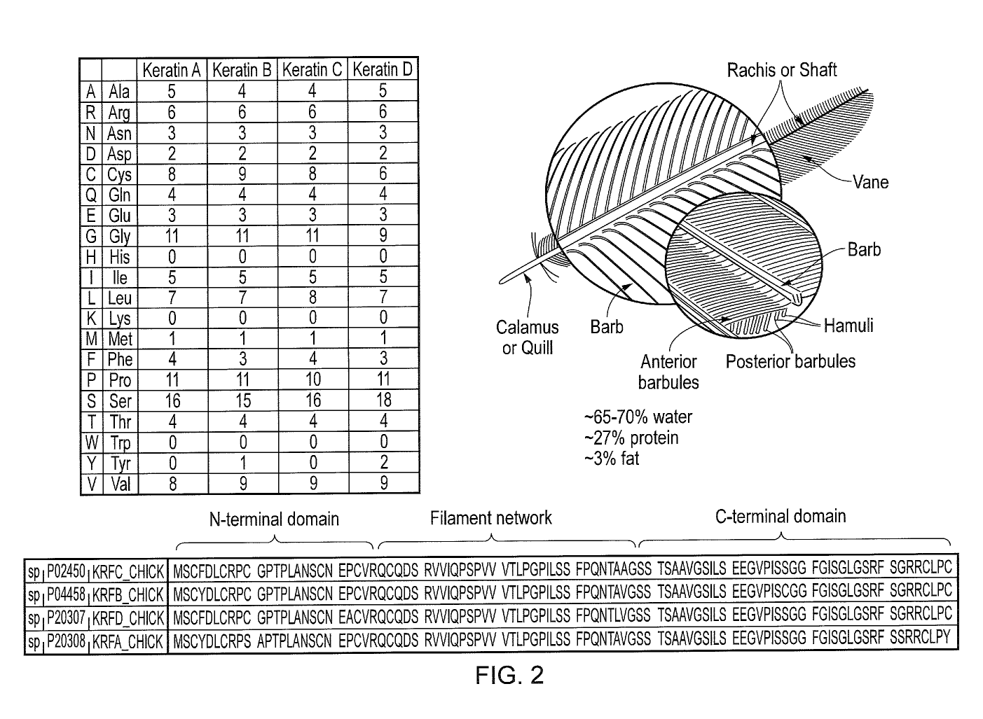

In one aspect, the present invention provides methods, apparatuses, compositions and kits for the enzymatic hydrolysis of feathers, e.g. chicken feathers. FIG. 2 depicts chicken feathers as an enzymatic substrate.

### Proteolytic Enzymes

In one aspect, the present invention is directed to systems, compositions and methods for converting keratin-containing proteinaceous material into a desirable food/feed product ingredient that is nutritious, palatable, and highly digestible by an animal comprising adding one or more enzymes during the process. In one embodiment, the present invention provides compositions and methods for converting keratin-containing proteinaceous material into a desirable food/feed product comprising adding one or more proteases alone or in combination with other enzymes during the process.

The term “protease” as used herein is synonymous with peptidase or proteinase.

The protease for use in the present invention may be a subtilisin (E.C. 3.4.21.62) or a bacillolysin (E.C. 3.4.24.28) or an alkaline serine protease (E.C. 3.4.21.x) or a keratinase (E.C. 3.4.x.x). Suitably, a protease for use in the present invention may be an Protease endopeptidase K (EC 3.4.2.1.64), pronase, papain, an endopeptidase Arg-C, an endoprotease Gluc-C (EC 3.4.21.19), an enterokinase (EC 3.4.21.9), a collagenase (EC 3.4.24.3), a thermolysin (EC 3.4.24.27), a trypsin (EC 3.4.21.4), a chymotrypsin (EC 3.4.21.1), a pepsin (EC 3.4.23.1), an aspergillopepsin (EC 3.4.23.18), a sedolisin (EC 3.4.21.100), or a dipeptidyl peptidase (EC 3.4.14.1).

Preferably the protease in accordance with the present invention is a subtilisin, a serine protease, a metalloprotease, an acid protease, a neutral protease or a keratinase.

Suitable proteases include those of animal, vegetable or microbial origin. Chemically modified or protein engineered mutants are also suitable. The protease may be a serine protease or a metalloprotease, e.g., an alkaline microbial protease or a trypsin-like protease. Examples of alkaline proteases are subtilisins, especially those derived from Bacillus sp., e.g., subtilisin Novo, subtilisin Carlsberg, subtilisin 309 (see, e.g., U.S. Pat. No. 6,287,841), subtilisin 147, and subtilisin 168 (see, e.g., WO 89/06279). Examples of trypsin-like proteases are trypsin (e.g., of porcine or bovine origin), and Fusarium proteases (see, e.g., WO 89/06270 and WO 94/25583). Examples of useful proteases also include but are not limited to the variants described in WO 92/19729 and WO 98/20115. All of which are incorporated herein by reference.

In one preferred embodiment the protease for use in the present invention may be one or more of the proteases in one or more of the commercial products below:

| Commercial | | | |
|---|---|---|---|
| product ® | Company | Protease type | Protease source |
| Protex 30L ™ | Genencor/DuPont | Serine protease* | B. subtilis |
| Protex 6L ™ | Genencor/DuPont | Serine protease* | B. amyloliquefaciens |
| Purafect 4000L ™ | Genencor/DuPont | Serine protease* | |
| FNA ™ | Genencor/DuPont | Serine protease* | B. amyloliquefaciens |
| Properase 1600L ™ | Genencor/DuPont | Serine protease* | B. alcalophilus |
| FoodPro PXT ™ | Genencor/DuPont | subtilisin | B. lentus |
| Esperase 8.0L | Novozymes | protease | Bacillus sp. |
| Everlase 16.0 ™ | | subtilisin | Bacillus sp. |
| Alcalase 2.4 ™ | Novozymes | subtilisin | Bacillus sp. |
| Neutrase 0.8L ™ | Novozymes | protease | B. amyloliquefaciens |
| Allzyme FD ™ | Alltech | Serine protease* | Aspergillus niger |
| Arazyme One-Q ™ | Insect Biotech Co. | metalloprotease | Serratia |
| | | | proteamacula ns HY-3 |
| Savinase ™ | Novozymes | subtilisin | Bacillus sp. |
| Ronozyme ProAct | DSM/Novozymes | Alkaline serine | Nocardiopsis prasina |
| | | protease | gene expressed in |
| | | | Bacillus |
| | | | licheniformis |
| Valkerase/Cibenza | Novus | Keratinase | Bacillus |
| IND900 | | | licheniformis |

Additionally or in the alternative the protease may be comprised in one or more of the following commercially available products: Kannase,™, NovoCarne Tender™, and Novozym 37020, Novo-Pro D™ (all available from Novozymes); BioSorb-ACDP™ (Noor Creations, India); or Angel™ Acid Protease (Angel Yeast Co, Ltd., China)

Suitably, the protease may be a protease from Bacillus (such as Bacillus subtilis, Bacillus amyloliquefaciens, B. alcalophilus and B. licheniformis), Trichoderma, Nocardiopsis, Serratia or Aspergillus.

In one embodiment, the protease is from Bacillus. Suitably, the protease may be from the species Bacillus subtilis, Bacillus amyloliquefaciens, B. alcalophilus, B. lentus and B. licheniformis. In one embodiment, the protease is from the species Bacillus subtilis.

In one embodiment, the protease has the amino acid SEQ ID No.1. In some embodiments, the protease has at least 80, 81, 82, 83, 84, 85, 86, 87, 88, 89, 90, 91, 92, 93, 94, 95, 96, 97, 98 or 99% identity with the amino acid SEQ ID No.1.

In one embodiment, the protease has the amino acid SEQ ID No.2. In some embodiments, the protease has at least 80, 81, 82, 83, 84, 85, 86, 87, 88, 89, 90, 91, 92, 93, 94, 95, 96, 97, 98 or 99% identity with the amino acid SEQ ID No.2.

In some embodiments, the enzyme has a total number of amino acids of less than 350, such as less than 340, such as less than 330, such as less than 320, such as less than 310, such as less than 300 amino acids, such as in the range of 200 to 350, such as in the range of 220 to 345 amino acids than the one or more enzyme described herein.

In some embodiments, the amino acid sequence of the enzyme has at least one, two, three, four, five, six, seven, eight, nine or ten amino acid substitutions.

The amino acid sequence may be prepared/isolated from a suitable source, or it may be made synthetically or it may be prepared by use of recombinant DNA techniques.

The protein encompassed in the present invention may be used in conjunction with other proteins, particularly enzymes. Thus the present invention also covers a combination of proteins wherein the combination comprises the protease of the present invention and another enzyme, which may be another protease according to the present invention.

Preferably the amino acid sequence when relating to and when encompassed by the per se scope of the present invention is not a native enzyme. In this regard, the term “native enzyme” means an entire enzyme that is in its native environment and when it has been expressed by its native nucleotide sequence.

### Compositions

In one aspect, the present disclosure provides compositions for converting keratin-containing proteinaceous material into a desirable food/feed product ingredient that is nutritious, palatable, and highly digestible by an animal. In some embodiments, the present invention relates to compositions for processing of keratin-containing proteinaceous material, in particular feathers, into a desirable food/feed product ingredient that is nutritious, palatable, and highly digestible by an animal. In one embodiment, the present invention provides compositions for converting keratin-containing proteinaceous material into a desirable food/feed product comprising one or more proteases alone or in combination with other enzymes. In some embodiments, the present invention provides compositions for improved processing proteinaceous material, e.g., reduced cost, increased digestibility and/or increase palatability of the protein hydrolysates.

In one aspect, the present invention is directed to composition comprising one or more enzymes for the processing of keratin-containing proteinaceous material a first step of reducing the size of the keratin-containing material (e.g. raw materials such as feathers) to a desire average particle size. In some embodiments, the process also involves steam or heat hydrolysis prior or after the enzyme hydrolysis. In some embodiments, the process also involves steam or heat hydrolysis after the enzyme hydrolysis. In some embodiments, the steam or heat hydrolysis occurs prior to the enzyme hydrolysis step. In some embodiments, the steam or heat hydrolysis occurs simultaneously with the size-reducing step prior to the enzyme hydrolysis step. The protein hydrolysate may be then further subjected to further processes to produce a food or feed ingredient.

In some embodiments, the present invention is directed to composition comprising one or more proteolytic enzymes. In some embodiments, the proteolytic enzyme is a subtilisin, a serine protease, a metalloprotease, an acid protease, or a neutral protease.

In some embodiments, the compositions comprise a proteolytic enzyme having at least 80, 81, 82, 83, 84, 85, 86, 87, 88, 89, 90, 91, 92, 93, 94, 95, 96, 97, 98 or 99% identity with the amino acid SEQ ID No.1. In one embodiment, the proteolytic enzyme has at least 85% identity with the amino acid SEQ ID No. 1. In one embodiment, the proteolytic enzyme has at least 90% identity with the amino acid SEQ ID No. 1. In one embodiment, the proteolytic enzyme has at least 95% identity with the amino acid SEQ ID No. 1. In some embodiments, the proteolytic enzyme has the amino acid SEQ ID No.1.

In some embodiments, the compositions comprise a proteolytic enzyme having at least 80, 81, 82, 83, 84, 85, 86, 87, 88, 89, 90, 91, 92, 93, 94, 95, 96, 97, 98 or 99% identity with the amino acid SEQ ID No.2. In one embodiment, the proteolytic enzyme has at least 85% identity with the amino acid SEQ ID No. 2. In one embodiment, the proteolytic enzyme has at least 90% identity with the amino acid SEQ ID No. 2. In one embodiment, the proteolytic enzyme has at least 95% identity with the amino acid SEQ ID No. 2. In one embodiment, the proteolytic enzyme has the amino acid SEQ ID No.2.

In some embodiments the enzyme composition according to the invention comprises a protease activity of at least about 1500 U/g, such as at least about 2000 U/g, such as at least about 2500 U/g, such as at least about 2800 U/g, such as at least about 3000 U/g, such as at least about 3200 U/g, such as at least about 3300 U/g, such as at least about 3500 U/g, such as at least about 3600 U/g as measured by in the assays described herein or any suitable assay known in the art. In some embodiments the enzyme composition according to the invention comprises a protease activity of at least about 2500 U/g to about 3600 U/g. In some embodiments the enzyme composition according to the invention comprises a protease activity of at least about 2800 U/g to about 3200 U/g.

In some embodiments, an enzyme composition comprising a proteolytic enzyme is added during the enzymatic hydrolysis process at a concentration of about 0.1 g/kg (proteolytic enzyme/keratin material) to about 10 g/kg. In some embodiments, an enzyme composition comprising a proteolytic enzyme is added during the enzymatic hydrolysis process at a concentration of about 0.3 g/kg to about 9 g/kg (proteolytic enzyme/keratin material). In some embodiments, the keratin material comprises (or consists of) feathers. In some embodiments the enzyme composition according to the invention comprises a protease activity of at least about 2800 U/g to about 3600 U/g.

In some embodiments, an enzyme composition comprising a proteolytic enzyme is added during the enzymatic hydrolysis process at a concentration of about 1 g/kg of keratin material to about 50 g/kg keratin material. In some embodiments, the keratin material comprises (or consists of) of wool, horns, hooves or admixtures thereof.

It will be understood that one protease unit (PU) is the amount of enzyme that liberates from the substrate (0.6% casein solution) one microgram of phenolic compound (expressed as tyrosine equivalents) in one minute at pH 7.5 (40 mM Na₂PO₄/lactic acid buffer) and 40° C. This may be referred to as the assay for determining 1 PU.

In some embodiments embodiment, the compositions comprise a subtilisin (E.C. 3.4.21.62) or a bacillolysin (E.C. 3.4.24.28) or an alkaline serine protease (E.C. 3.4.21.x) or a keratinase (E.C. 3.4.x.x) and the E.C. classification designates an enzyme having that activity when tested in the assay taught herein for determining 1 PU.

In some embodiments, the compositions comprise a protease that is an alkaliphile and optimally hydrolyses at a pH between about pH 7 to about pH 12. Suitably, the protease may optimally hydrolyse at a pH between about pH 8 to about pH 11. Suitably, the protease may optimally hydrolyse at a pH between about pH 8 to about pH 10. Suitably, the protease may optimally hydrolyse at a pH of about 9.

In some embodiments, the compositions comprise a protease that is an acidophile and optimally hydrolyses at a pH between about pH 1 to about pH 7. Suitably, the protease may optimally hydrolyse at a pH between about pH 3 to about pH 6. Suitably, the protease may optimally hydrolyse at a pH between about pH 4 to about pH 5.

In some embodiments, the compositions comprise a protease that is a neutrophile and optimally hydrolyses at a pH between about pH 6 to about pH 8. Suitably, the protease may optimally hydrolyse at a pH of about 7.

In some embodiments, the proteases may hydrolyse optimally at a temperature between about 30° C. to about 98° C. Suitably, the protease may hydrolyse optimally at a temperature between about 40° C. to about 80° C. Suitably, the protease may hydrolyse optimally at a temperature between about 50° C. to about 80° C. Preferably, the protease may hydrolyse optimally at a temperature between about 60° C. to about 80° C.

The enzyme compositions used in the methods according to the present invention may be compositions produced by fermentation of selected fungal or bacterial strains, such as Trichoderma reesei or Bacillus subtilis. Enzyme compositions may contain water, stabilizing agents, such as sorbitol, and salts, such as sodium chloride, sodium benzoate, and potassium sorbate with a pH in the range of 4-6, such as 4.5-5. The enzyme compositions may be exempt from FDA labelling and approved for food products.

In some embodiments, the enzyme compositions are liquid compositions. Preferred liquid enzymatic products are produced by using selected strains of bacteria or fungi. In some embodiments, the liquid compositions comprising the enzymes described herein easily mix and combine with the keratin-containing proteinaceous material. In some embodiments, the liquid compositions comprise the enzymes exhibiting proteolyic activities at an amount of about 2800 U/g to about 3600 U/g. In some embodiments, the liquid composition comprises other enzymes selected from the group consisting of cellulase, mannanase, xylanase, amylase, lipase and a combination thereof.

In some embodiments, the proteolytic enzyme is present in an amount of 0.1 kg-30 kg of enzyme per MT of keratin-containing proteinaceous material, e.g., feathers. In some embodiments the moisture content of the keratin containing material (e.g. feathers) is about 65 to 70%. In some embodiments, the proteolytic enzyme has a protease activity of at least about 2800 U/g to about 3600 U/g.

Materials may be added to an enzyme-containing liquid to improve the properties of the liquid composition. Non-limiting examples of such additives include: salts (e.g., alkali salts, earth metal salts, additional chloride salts, sulfate salts, nitrate salts, carbonate salts, where exemplary counterions are calcium, potassium, and sodium), inorganic minerals or clays (e.g., zeolites, kaolin, bentonite, talc's and/or silicates), carbohydrates (e.g., sucrose and/or starch), coloring pigments (e.g., titanium dioxide), biocides (e.g., Rodalon®, Proxel®), dispersants, anti-foaming agents, reducing agents, acid agents, alkaline agents, enzyme stabilizers (e.g. polyol such as glycerol, propylene glycol, sorbitol, inorganic salts, sugars, sugar or a sugar alcohol, lactic acid, boric acid, or a boric acid derivative and combinations thereof), enzyme inhibitors, preservative (e.g. methyl paraben, propyl paraben, benzoate, sorbate or other food approved preservatives) and combinations thereof. Excipients which may be used in the preparation/composition include maltose, sucrose, glucose including glucose syrup or dried glucose syrup, pre-cooked starch, gelatinised starch, L-lactic, ascorbyl palmitate, tocopherols, lecithins, citric acid, citrates, phosphoric, phosphates, sodium alginate, carrageenan, locust bean gum, guar gum, xanthan gum, pectins, sodium carboxymethylcellulose, mono- and diglycerides, citric acid esters of mono- and diglycerides, sucrose esters, carbon dioxide, argon, helium, nitrogen, nitrous oxide, oxygen, hydrogen, and starch sodium octenylsuccinate.

### Methods

In one aspect, the present disclosure provides methods for converting keratin-containing proteinaceous material into a desirable food/feed product ingredient that is nutritious, palatable, and highly digestible by an animal. In some embodiments, the present invention relates to methods for processing of keratin-containing proteinaceous material, in particular feathers, into a desirable food/feed product ingredient that is nutritious, palatable, and highly digestible by an animal. In one embodiment, the present invention provides methods for converting keratin-containing proteinaceous material into a desirable food/feed product comprising one or more proteases alone or in combination with other enzymes. In some embodiments, the present invention provides compositions for improved processing proteinaceous material, e.g., reduced cost, increased digestibility and/or increase palatability of the protein hydrolysates.

The term “keratin hydrolysate” or “protein hydrolysates” as used herein refers to the resultant product following the hydrolysis of a keratin material by e.g. a protease.

The present invention provides methods for preparing food protein ingredients that involves a first step of reducing the size of the keratin-containing material (e.g. raw materials such as feathers) to a desire average particle size. The sized-reduced keratin material is then subjected to enzyme hydrolysis by mixing a quantity of at least one proteolytic enzyme with the pretreated sized-reduced keratin material. In some embodiments, the process also involves steam or heat hydrolysis prior or after the enzyme hydrolysis. In some embodiments, the steam or heat hydrolysis occurs prior to the enzyme hydrolysis step. In some embodiments, the steam or heat hydrolysis occurs simultaneously with the size-reducing step prior to the enzyme hydrolysis step. The protein hydrolysate may be then further subjected to further processes to produce a food or feed ingredient. In some embodiments, the invention provides a process that does not need the use of chemicals for chemical hydrolysis of the peptides.

The size reduction step can include any conventional process for reducing the size. Preferred size-reduction processes are selected from the group consisting of grinding, milling, chopping, cutting, dicing, shredding, emulsifying, homogenizing, high pressure homogenizing and combinations thereof. In some embodiments, the keratin-containing material (e.g. raw materials such as feathers) is reduced to an average particle size of about 5 mm or less, or 4.5 mm or less, or less than 4 mm, 3 mm, 2 mm, 1 mm, 0.9 mm, 0.8 mm, 0.7 mm, 0.6 mm, or 0.5 mm, 0.4 mm, 0.3 mm, 0.2 mm, 0.1 mm, or 0.05 mm. In some embodiments, the keratin-containing material (e.g. raw materials such as feathers) is reduced to an average particle size of less than 50 nm, 40 nm, 30 nm, 20 nm, 10 nm, 15 nm, 14 nm, 13 nm, 12 nm, 11 nm, 10 nm, 9 nm, 8 nm, 7 nm, 6 nm, 5 nm, 4 nm, 3 nm, 2 nm, or 1 nm.

In some embodiments, the methods includes the steps of (a) processing a keratin-containing material (e.g. feathers) through a size-reduction process to produce a size-reduced keratin material, wherein said reduced keratin material comprises an average particle size of 5 mm or less, (b) admixing with said size-reduced keratin material with one or more proteases, and (c) hydrolyzing said keratin material by heat/steam hydrolysis, wherein step (b) and (c) can occur in any order. In some embodiments, the steam or heat hydrolysis occurs prior to the enzyme hydrolysis step. In some embodiments, the steam or heat hydrolysis occurs simultaneously with the size-reducing step prior to the enzyme hydrolysis step. In some embodiments, the keratin-containing material (e.g. raw materials such as feathers) is reduced to an average particle size of about 5 mm or less, or 4.5 mm or less, or less than 4 mm, 3 mm, 2 mm, 1 mm, 0.9 mm, 0.8 mm, 0.7 mm, 0.6 mm, or 0.5 mm, 0.4 mm, 0.3 mm, 0.2 mm, 0.1 mm, or 0.05 mm. In some embodiments, the keratin-containing material (e.g. raw materials such as feathers) is reduced to an average particle size of less than 50 nm, 40 nm, 30 nm, 20 nm, 10 nm, 15 nm, 14 nm, 13 nm, 12 nm, 11 nm, 10 nm, 9 nm, 8 nm, 7 nm, 6 nm, 5 nm, 4 nm, 3 nm, 2 nm, or 1 nm. In some embodiments, the keratin-containing material is feathers. In some embodiments, the protease has at least 80, 81, 82, 83, 84, 85, 86, 87, 88, 89, 90, 91, 92, 93, 94, 95, 96, 97, 98 or 99% identity with the amino acid SEQ ID No.1. In one embodiment, the proteolytic enzyme has at least 85% identity with the amino acid SEQ ID No. 1. In one embodiment, the proteolytic enzyme has at least 90% identity with the amino acid SEQ ID No. 1. In one embodiment, the proteolytic enzyme has at least 95% identity with the amino acid SEQ ID No. 1. In some embodiments, protease has the amino acid SEQ ID No.1. In some embodiments, the protease has at least 80, 81, 82, 83, 84, 85, 86, 87, 88, 89, 90, 91, 92, 93, 94, 95, 96, 97, 98 or 99% identity with the amino acid SEQ ID No.2. In one embodiment, the proteolytic enzyme has at least 85% identity with the amino acid SEQ ID No. 2. In one embodiment, the proteolytic enzyme has at least 90% identity with the amino acid SEQ ID No. 2. In one embodiment, the proteolytic enzyme has at least 95% identity with the amino acid SEQ ID No. 2. In some embodiments, protease has the amino acid SEQ ID No.2.

In some embodiments, the invention provides a process that does not need the use of chemicals for chemical hydrolysis of the peptides. The present inventors surprisingly found an improved process providing steps to loosen tightly-packed beta-sheet structure, and to allow enzymatic hydrolysis to efficiently break the peptide bonds of the keratin without the need to use and extra step for chemical hydrolysis. The present inventors surprisingly found that the methods described herein provide protein hydrolysates with increased palatability and digestibility. In some embodiments, the methods described herein provide protein hydrolysates without the production of undesirable amino acids that result from chemical hydrolysis. Thus, in some embodiments, the method include the steps of (a) processing a keratin-containing material (e.g. feathers) through a size-reduction process to produce a size-reduced keratin material, wherein said reduced keratin material comprises an average particle size of 5 mm or less, (b) admixing with said size-reduced keratin material with one or more proteases, and (c) hydrolyzing said keratin material by heat/steam hydrolysis, wherein step (b) and (c) can occur in any order, wherein no further chemical hydrolysis of the peptides is performed. In some embodiments, the protease is a subtilisin, a serine protease, a metalloprotease, an acid protease, or a neutral protease. In some embodiments, the protease has at least 80, 81, 82, 83, 84, 85, 86, 87, 88, 89, 90, 91, 92, 93, 94, 95, 96, 97, 98 or 99% identity with the amino acid SEQ ID No.1. In one embodiment, the proteolytic enzyme has at least 85% identity with the amino acid SEQ ID No. 1. In one embodiment, the proteolytic enzyme has at least 90% identity with the amino acid SEQ ID No. 1. In one embodiment, the proteolytic enzyme has at least 95% identity with the amino acid SEQ ID No. 1. In some embodiments, protease has the amino acid SEQ ID No.1. In some embodiments, the protease has at least 80, 81, 82, 83, 84, 85, 86, 87, 88, 89, 90, 91, 92, 93, 94, 95, 96, 97, 98 or 99% identity with the amino acid SEQ ID No.2. In one embodiment, the proteolytic enzyme has at least 85% identity with the amino acid SEQ ID No. 2. In one embodiment, the proteolytic enzyme has at least 90% identity with the amino acid SEQ ID No. 2. In one embodiment, the proteolytic enzyme has at least 95% identity with the amino acid SEQ ID No. 2. In some embodiments, protease has the amino acid SEQ ID No.2.

In some embodiments, the methods includes the steps of (a) processing a keratin-containing material (e.g. feathers) through a size-reduction process to produce a size-reduced keratin material, wherein said reduced keratin material comprises an average particle size of 5 mm or less, (b) hydrolyzing said keratin material by heat/steam hydrolysis, and (c) admixing with said size-reduced keratin material with one or more proteases. In some embodiments, no further chemical hydrolysis of the peptides is performed. In some embodiments, the steam or heat hydrolysis occurs simultaneously with the size-reducing step. In some embodiments, the protease is a subtilisin, a serine protease, a metalloprotease, an acid protease, or a neutral protease. In some embodiments, the protease has at least 80, 81, 82, 83, 84, 85, 86, 87, 88, 89, 90, 91, 92, 93, 94, 95, 96, 97, 98 or 99% identity with the amino acid SEQ ID No.1. In one embodiment, the proteolytic enzyme has at least 85% identity with the amino acid SEQ ID No. 1. In one embodiment, the proteolytic enzyme has at least 90% identity with the amino acid SEQ ID No. 1. In one embodiment, the proteolytic enzyme has at least 95% identity with the amino acid SEQ ID No. 1. In some embodiments, protease has the amino acid SEQ ID No.1. In some embodiments, the protease has at least 80, 81, 82, 83, 84, 85, 86, 87, 88, 89, 90, 91, 92, 93, 94, 95, 96, 97, 98 or 99% identity with the amino acid SEQ ID No.2. In one embodiment, the proteolytic enzyme has at least 85% identity with the amino acid SEQ ID No. 2. In one embodiment, the proteolytic enzyme has at least 90% identity with the amino acid SEQ ID No. 2. In one embodiment, the proteolytic enzyme has at least 95% identity with the amino acid SEQ ID No. 2. In some embodiments, protease has the amino acid SEQ ID No.2.

In some embodiments, the methods includes the steps of a) processing a keratin-containing material (e.g. feathers) through a size-reduction process to produce a size-reduced keratin material, wherein said reduced keratin material comprises an average particle size of 5 mm or less, (b) admixing with said size-reduced keratin material with one or more proteases, and (c) hydrolyzing said keratin material by heat/steam hydrolysis. In some embodiments, no further chemical hydrolysis of the peptides is performed. In some embodiments, the protease is a subtilisin, a serine protease, a metalloprotease, an acid protease, or a neutral protease. In some embodiments, the protease has at least 80, 81, 82, 83, 84, 85, 86, 87, 88, 89, 90, 91, 92, 93, 94, 95, 96, 97, 98 or 99% identity with the amino acid SEQ ID No.1. In one embodiment, the proteolytic enzyme has at least 85% identity with the amino acid SEQ ID No. 1. In one embodiment, the proteolytic enzyme has at least 90% identity with the amino acid SEQ ID No. 1. In one embodiment, the proteolytic enzyme has at least 95% identity with the amino acid SEQ ID No. 1. In some embodiments, protease has the amino acid SEQ ID No.1. In some embodiments, the protease has at least 80, 81, 82, 83, 84, 85, 86, 87, 88, 89, 90, 91, 92, 93, 94, 95, 96, 97, 98 or 99% identity with the amino acid SEQ ID No.2. In one embodiment, the proteolytic enzyme has at least 85% identity with the amino acid SEQ ID No. 2. In one embodiment, the proteolytic enzyme has at least 90% identity with the amino acid SEQ ID No. 2. In one embodiment, the proteolytic enzyme has at least 95% identity with the amino acid SEQ ID No. 2. In some embodiments, protease has the amino acid SEQ ID No.2.

Steam is a preferred heat source for the heat hydrolysis step, although it is foreseen that other suitable means of heat known in the industry could also be employed. Steam heat allows for greater penetration of the heat within the keratin-containing proteinaceous material. The heat is also important to reduce or eliminate microbial pathogens found on or within the proteinaceous material. By limiting the heat energy input of the reaction, the steam also serves to reduce the production of detrimental amino acids. In some embodiments, low pressure, high volume steam is used, particularly highly saturated steam, as it increases heat transfer and allows minimal high temperature exposure. In other embodiments, superheated steam treatment may also be used.

In some embodiments, the steam has a temperature of from about 100° C. to about 160° C., or about 100° C. to about 130° C., or about, 120° C. to about 140° C., or about 148° C. to about 157° C., and may be introduced via a perforated plate at the bottom of a tank or in any other manner designed to enable the steam to contact and penetrate the mass of keratin-containing material. In some embodiments, the steam has a temperature of from about 120° C. to about 130° C. Steam is supplied to the keratin-containing material reaches a temperature of from about 85° C. to about 95° C. In some embodiments, the size-reducing step occurs simultaneously with the heat hydrolysis step. In some embodiments, the keratin-containing material can be agitated, such as by stirring or grinding to allow for size-reduction of the keratin-containing material. In some embodiments, the keratin-containing material (e.g. raw materials such as feathers) is reduced to an average particle size of about 5 mm or less, or 4.5 mm or less, or less than 4 mm, 3 mm, 2 mm, 1 mm, 0.9 mm, 0.8 mm, 0.7 mm, 0.6 mm, or 0.5 mm, 0.4 mm, 0.3 mm, 0.2 mm, 0.1 mm, or 0.05 mm. In some embodiments, the keratin-containing material (e.g. raw materials such as feathers) is reduced to an average particle size of less than 50 nm, 40 nm, 30 nm, 20 nm, 10 nm, 15 nm, 14 nm, 13 nm, 12 nm, 11 nm, 10 nm, 9 nm, 8 nm, 7 nm, 6 nm, 5 nm, 4 nm, 3 nm, 2 nm, or 1 nm. In some embodiments, the keratin-containing material is feathers.

In some embodiments, heat is supplied for a period of from about 5 to about 30 minutes and the high temperature of the heated material can be maintained by insulation methods before the next process step. It is foreseen that the effects of the steam may be maintained by insulation methods for a period of up to about 2.0 hours. In another embodiment heat is generally supplied from about 15 to about 30 minutes. The duration of the heat treatment will depend on a number of processing variables, such as, but not limited to, the heating method, the containment vessel, agitation or grinding during the heating step, the transportation period before the next process step, and whether other agents are used (e.g. detergent and/or pH balancing agents). In another embodiment, it is also foreseen that the pretreatment denaturation step could be conducted under non-ambient conditions, such as in a low oxygen environment or under elevated pressure.

The admixture step with one or more proteases may occur in a vessel or reactor. In one embodiment, the vessel or reactor is a free-fall vessel or reactor. Examples of free-fall vessels include drum mixers and tumble mixers.

Suitably the keratin material and the protease may optionally be admixed simultaneously or sequentially) with one or more further components.

The keratin material and protease are admixed until a desired degree of degradation of the keratin material has occurred (e.g. until the digestibility of keratin material is increased thereby enriching the concentration of digestible proteins and peptides therein). A person of ordinary skill in the art will readily understand that the optimal time period used will depend on a number of factors such as the temperature and pH used; the degree of cross-linking present in the keratin material to be degraded; whether other components such as non-sulphur containing surfactants, chemical oxidants, acids or alkalis are used in the process and the ratio of reducing agent and/or protease to keratin material for example.

In one embodiment, the keratin material and protease may be admixed for about 30 minutes to about 48 hours. Suitably, the keratin material and protease may be admixed from about 1 hour to about 42 hours; or from about 2 hours to about 36 hours; or from about 3 hours to about 30 hours; or from about 4 hours to about 24 hours; or from about 5 to about 18 hours.

In one embodiment, the keratin material and protease may be admixed for about 30 minutes to about 16 hours. In one embodiment, the keratin material and protease may be admixed for about 30 minutes to about 14 hours or from about 1 hour to about 12 hours or from about 1.5 hours to about 10 hours or from about 2 hours to about 8 hours or from about 3 to about 7 hours.

Suitably the keratin material and protease may be admixed for at least 30 minutes. Suitably the keratin material and protease may be admixed for at least 45 minutes or at least 1 hour or at least 1.5 hours or at least 2 hours or at least 2.5 hours or at least 3 hours or at least 3.5 hours or at least 4 hours or at least 4.5 hours or at least 5 hours or at least 5.5 hours or at least 6 hours or at least 7 hours or at least 8 hours or at least 9 hours or at least 10 hours.

In one embodiment, the keratin material and protease is admixed for at least 2 hours.

Suitably, the keratin material and protease may be admixed for less than 16 hours, less than 15.5 hours, less than 15 hours, less than 14.5 hours, less than 14 hours or less than 13 hours.

In one embodiment, the keratin and material and protease may be admixed for less than 16 hours.

In one embodiment, the keratin and material and protease may be admixed for less than 13 hours.

In one embodiment, the keratin and material and protease may be admixed for about 1.5 to about 10 hours.

In one embodiment, the keratin material and protease may be admixed until at least 50% by weight of the keratin material is degraded. Optionally, the keratin material and protease may be admixed until at least 55% (suitably at least 60% or at least 70% or at least 80% or at least 90% or 100%) by weight of the keratin material is degraded. Degraded or degradation of feather material by 100% may be defined by the complete detachment of vanes, barbs and after feather from the rachis and hollow shaft; and the fragmentation of the rachis and hallow shaft such that feather meal is produced in one single step.

In some embodiments, the protease may be added with a reducing agent (e.g. sulfite).

In some embodiments, the keratin material may be added to a vessel (e.g. at ambient temperature) and heated (for example to 50-80° C.) with the addition of a protease, and optionally a reducing agent (e.g. sulfite). In some embodiments, the keratin material may be added to a vessel (e.g. at ambient temperature) and heated (for example to 50-80° C.) with the addition of a protease, a chemical and optionally a reducing agent (e.g. sulfite). The vessel may be a closed reactor optionally with reduced airspace to control oxygen levels. The keratin material and protease are reacted for a suitable time (e.g. for 30 min to 48 hours). The vessel may be rotated or the vessel contents may be admixed (e.g. using a propeller at 1-200 rpm). During the reaction process the mixing may cause some oxygen to be continually introduced into to the reaction liquid. Accordingly, the airspace of the vessel may advantageously have lower level of oxygen (e.g. through the use of stream to expel air out of the reactor).

Suitably the pH during the reaction is within the working range of the protease used. It is a matter of routine to a person of ordinary skill in the art to determine the optimal working range of the protease used and to add buffer to adjust the reaction solution to an appropriate pH. For example, the working range of the protease Protex 30L (available from DuPont Industrial Biosciences ApS) may be from about 5.5 to about 12. When Protex 30 L is used as the protease, the pH during the admixing step may be from about 5.5 to about 12. Suitably, the pH range may be from about 7 to about 11, or from about 8 to about 10. Preferably, when Protex 30L is used the pH is about pH 9.

In one embodiment, the pH used is pH at about the optimal pH for the protease used. For example, the pH may be +/− about 1 pH of the optimal pH of the protease used (e.g. a pH of about 8 to about 10 for Protex 30L). Suitably, the pH may be +/− about 0.5 pH of the optimal pH of the protease used or about the optimal pH.

In another embodiment, the pH used is the optimal pH of the protease.

In some embodiments, the pH during the heat hydrolysis step is adjusted to +/− about 0.5-1 pH of the optimal pH of the protease at any time during the process. In some embodiments, the heat hydrolysis step is carried out at +/− about 0.5-1 pH of the optimal pH of the protease.

Suitably the temperature during the reaction is adjusted to be within the working range of the protease used. It is a matter of routine to a person of ordinary skill in the art to determine the optimal working range of the protease used and to carry out the reaction at a desired temperature. For example, the working range of the protease Protex 30L may be from about 30° C. to about 80° C. When Protex 30 L is used as the protease, the temperature during the admixing step may be from about 30° C. to about 80° C. Suitably, the temperature may be from about 40° C. to about 80° C., or from about 50° C. to about 80° C. Suitably, the temperature may be or from about 60° C. to about 80° C. Preferably, when Protex 30L is used the temperature is about 70° C.

Suitably the temperature used may be + and/or −15° C. or + and/or −10° C.; or + and/or −5° C.; or + and/or −4° C.; or + and/or −3° C.; or + and/or −2° C. or + and/or −1° C. of the optimal temperature of the protease used. Preferably the temperature used is about the optimal working temperature of the protease used.

In one embodiment, the temperature used is + and/or −10° C. of the optimal temperature of the protease used.

In another embodiment, the temperature used is + and/or −5° C. of the optimal temperature of the protease used.

In one embodiment, more than one enzyme may be present in the reaction (e.g. admixing) step. Suitably, the temperature, pH and other reaction conditions used are selected to be within the working ranges of the enzymes used. Suitably, the enzymes used (e.g. more than one protease and/or other additional enzymes) are selected which have overlapping working ranges. Preferably, the enzymes are selected to have compatible, preferably similar working ranges.

Suitably, prior to admixing the keratin material with the protease the feather may be sterilized e.g. to reduce and/or prevent bacterial contamination. This sterilization step may be carried out by any available means. For example, fumigation could be achieved by contacting the keratin material with formalin or ethylene oxide gas.

In one embodiment of the present invention, the methods of the present invention comprises a chemical hydrolysis step which may occur prior to, during and/or after the reaction step with protease. In one embodiment of the present invention, the methods of the present invention may comprise a chemical hydrolysis step occurs prior to and/or during the reaction step with protease. In one embodiment of the present invention, the methods of the present invention may comprise a chemical hydrolysis step occurs after the reaction step with protease. In one embodiment, suitably the acid or alkali used in the chemical hydrolysis may provide the means for pH adjustment to the optimal working conditions of the protease. In one embodiment, chemical hydrolysis may occur after the reaction step with a protease. The process of the present invention may be carried out as a batch, fed-batch or continuous process. Example of chemicals that can be used for chemical hydrolysis and chemical hydrolysis processes are described in PCT publication numbers WO2014/013081, WO2014/013080 and WO2014/013082, which are incorporated here by reference.

Suitably, the keratin material and protease and/or chemical may optionally be admixed (simultaneously or sequentially) with one or more further components. Examples of further components include reducing agents, surfactants, additional enzymes, antimicrobials, metal ions in the form of a salt, carriers, excipients, diluents, fats, peptides and minerals. Examples are described in PCT publication numbers WO2014/013081, WO2014/013080 and WO2014/013082, which are incorporated here by reference.

In some embodiments, the process of the present invention may be carried out in a batch process. Advantageously, a batch process is more easily adapted for controlling oxygen levels.

In some embodiments, the process of the present invention may be carried out in an oxygen controlled environment. Examples on how to control oxygen levels are described in described in PCT publication numbers WO2014/013081, WO2014/013080 and WO2014/013082, which are incorporated here by reference.

### Protein Hydrolysate

In one aspect, the present disclosure provides keratin hydrolysates that are nutritious, palatable, and highly digestible by an animal. In some embodiments, the keratin hydrolysates have improved texture and appeal. Without intending to be limited to any theory the present inventors surprisingly found compositions, methods and processes that result in the presence of soluble peptides that make the product more palatable, as the keratin hydrolysates have more taste active compounds. The present inventors surprisingly found compositions, methods and processes that result in the combination of correct peptide size and specificity of cleavage (e.g. nature of N-terminal amino acid) that produce keratin hydrolysates that are nutritious, palatable, and highly digestible by an animal.

In some embodiments, the keratin hydrolysates are highly palatable and contain from about 76% to about 100% protein digestibility. In another embodiment, the keratin hydrolysates contain from about 80% to about 100% protein digestibility. In other embodiments, the keratin hydrolysates contain from about 85%, 86%, 87%, 88%, 89%, 90%, 91%, 92%, 93%, 94%, 95%, 96%, 97%, 98%, or 99% to about 100% protein digestibility. In still other embodiments, the keratin hydrolysates contain at least about 93% protein digestibility.

In some embodiments, the keratin hydrolysates have at least about 5% sulfur-containing amino acids in total amino acids. At least about 90% of the keratin hydrolysates comprise short chain peptides, having a molecular weight of less than about 10 kDa. In some embodiments, at least about 80% of the keratin hydrolysates comprise peptides having a molecular weight in the range of from about 0.1 kDa to about 5 kDa, and preferably in the range of from about 0.1 kDa to about 1.0 kDa. In some embodiments, at least about 80% of the keratin hydrolysates comprise peptides having a molecular weight in the range of from about 0.1 kDa to about 0.9 kDa, and preferably in the range of from about 0.1 kDa to about 0.8 kDa. In some embodiments, the product contains a variety of available free amino acids, including but not limited to Leucine, Arginine, Glutamic acid, Glycine, Serine and Phenylalanine.

In some embodiments, the protein hydrolysate is rich in peptides with N-terminal Leucine, Isoleucine, Valine, Alanine, Tyrosine, Phenylalanine and Lysine. In some embodiments, the keratin hydrolysates have at least about 50% of peptides with N-terminal Leucine, Isoleucine, Valine, Alanine, Tyrosine, Phenylalanine or Lysine. In some embodiments, the keratin hydrolysates have at least about 60% of peptides with N-terminal Leucine, Isoleucine, Valine, Alanine, Tyrosine, Phenylalanine or Lysine. In some embodiments, the keratin hydrolysates have at least about 70% of peptides with N-terminal Leucine, Isoleucine, Valine, Alanine, Tyrosine, Phenylalanine or Lysine. In some embodiments, the keratin hydrolysates have at least about 80% of peptides with N-terminal Leucine, Isoleucine, Valine, Alanine, Tyrosine, Phenylalanine or Lysine. In some embodiments, the keratin hydrolysates have at least about 90% of peptides with N-terminal Leucine, Isoleucine, Valine, Alanine, Tyrosine, Phenylalanine or Lysine.

In some embodiments, the keratin hydrolysates have at least about 50% of peptides with N-terminal Leucine, Isoleucine, Valine, Alanine, Tyrosine, Phenylalanine or Lysine, and a molecular weight of about 0.1 kDa to about 1.0 kDa. In some embodiments, the keratin hydrolysates have at least about 50% of peptides with N-terminal Leucine, Isoleucine, Valine, Alanine, Tyrosine, Phenylalanine or Lysine, and a molecular weight of about in the range of from about 0.1 kDa to about 0.9 kDa, and preferably in the range of from about 0.1 kDa to about 0.8 kDa. In some embodiments, the keratin hydrolysates have at least about 70% of peptides with N-terminal Leucine, Isoleucine, Valine, Alanine, Tyrosine, Phenylalanine or Lysine, and a molecular weight of about 0.1 kDa to about 1.0 kDa. In some embodiments, the keratin hydrolysates have at least about 70% of peptides with N-terminal Leucine, Isoleucine, Valine, Alanine, Tyrosine, Phenylalanine or Lysine, and a molecular weight of about in the range of from about 0.1 kDa to about 0.9 kDa, and preferably in the range of from about 0.1 kDa to about 0.8 kDa. In some embodiments, the keratin hydrolysates have at least about 80% of peptides with N-terminal Leucine, Isoleucine, Valine, Alanine, Tyrosine, Phenylalanine or Lysine, and a molecular weight of about 0.1 kDa to about 1.0 kDa. In some embodiments, the keratin hydrolysates have at least about 80% of peptides with N-terminal Leucine, Isoleucine, Valine, Alanine, Tyrosine, Phenylalanine or Lysine, and a molecular weight of about in the range of from about 0.1 kDa to about 0.9 kDa, and preferably in the range of from about 0.1 kDa to about 0.8 kDa. In some embodiments, the keratin hydrolysates have at least about 90% of peptides with N-terminal Leucine, Isoleucine, Valine, Alanine, Tyrosine, Phenylalanine or Lysine, and a molecular weight of about 0.1 kDa to about 1.0 kDa. In some embodiments, the keratin hydrolysates have at least about 90% of peptides with N-terminal Leucine, Isoleucine, Valine, Alanine, Tyrosine, Phenylalanine or Lysine, and a molecular weight of about in the range of from about 0.1 kDa to about 0.9 kDa, and preferably in the range of from about 0.1 kDa to about 0.8 kDa.

In some embodiments, the protein hydrolysates have 50% less undesirable amino acids that result from chemical hydrolysis. In some embodiments, the protein hydrolysates have 70% less undesirable amino acids that result from chemical hydrolysis. In some embodiments, the protein hydrolysates have 90% less undesirable amino acids that result from chemical hydrolysis. In some embodiments, the protein hydrolysates are substantially free of undesirable amino acids that result from chemical hydrolysis.

In another aspect there is provided a method for producing animal feed comprising admixing keratin hydrolysate produced by a method of the present invention with one or more animal feed constituents. Examples of different type of animal feeds and their preparation are described in in PCT publication numbers WO2014/013081, WO2014/013080 and WO2014/013082, which are incorporated here by reference.

The keratin hydrolysate produced by the method of the present invention may be used in any suitable form—whether when alone or when present in a composition. Said composition may include other nutrition-rich waste streams from slaughter houses such as blood or meat and bone meal, or it may be prepared in a composition with other animal feeds including but not limited to soybean meal, fish meal, fish oil, whey powder, whey filtrate, distillers grains, cottonseed meal, corn gluten meal, canola meal, and the like.

The dry powder or granules may be prepared by means known to those skilled in the art, such as, in top-spray fluid bed coater, in a bottom spray Wurster or by drum granulation (e.g. High sheer granulation), extrusion, pan coating or in a microingredients mixer.

Suitably, the keratin hydrolysate may be provided as a spray-dried or freeze-dried powder. Such spray dried powders have the advantages of increased stability and handling compared to forms with a higher water content, but said powders are still able to be provided in solution or suspended form to young animals most in need of nutritional supplement. In one embodiment of the current invention the spray dried keratin hydrolysate is in a powder form especially suitable for feeding young pets and commercial livestock, including but not limited to cows, pigs, mink, dogs, cats, broiler chickens and turkeys. The small particle size of the spray dried keratin hydrolysate also makes it particularly suitable for smaller creatures such as young fish (fry), and crustaceans such as shrimp and crab larvae.

In one aspect, the keratin hydrolysate is in a liquid formulation. Such liquid consumption may contain one or more of the following: a buffer, salt, sorbitol and/or glycerol.

In one embodiment the keratin hydrolysate of the present invention may formulated with at least one physiologically acceptable carrier selected from at least one of maltodextrin, limestone (calcium carbonate), cyclodextrin, wheat or a wheat component, sucrose, starch, Na2SO4, Talc, PVA, sorbitol, benzoate, sorbiate, glycerol, sucrose, propylene glycol, 1,3-propane diol, glucose, parabens, sodium chloride, citrate, acetate, phosphate, calcium, metabisulfite, formate and mixtures thereof.

### Examples

The present disclosure is described in further detail in the following examples, which are not in any way intended to limit the scope of the disclosure as claimed. The attached figures are meant to be considered as integral parts of the specification and description of the disclosure. The following examples are offered to illustrate, but not to limit the claimed disclosure.

### Enzymatic Feather Hydrolysis

The goal is to develop an economically viable enzymatic feather hydrolysis process compared to the conventional one (high temperature, pressure).

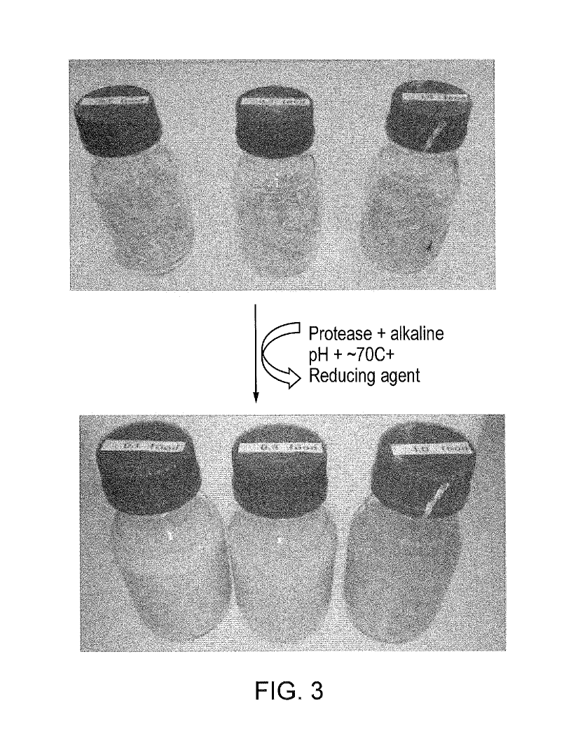

FIG. 3 shows a process and results for an enzymatic feather hydrolysis process.

The benchmark for a standard feather meal (high pressure, temperature) is >75% in Pepsin-HCl digestibility. Thus, the goal is to reach >75% using a cost-viable enzyme solution.

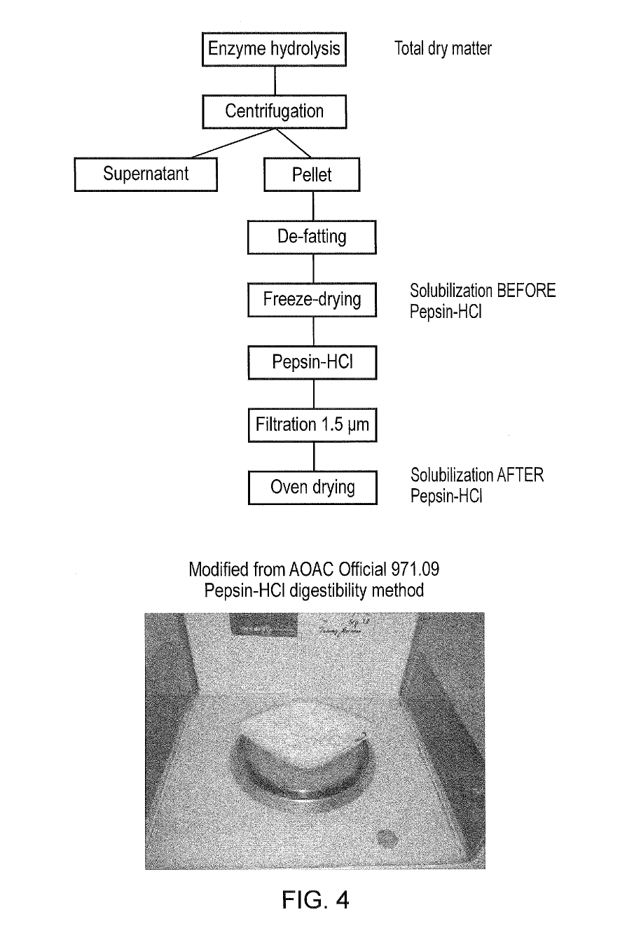

FIG. 4 shows a method for evaluating pepsin-HCl digestibility.

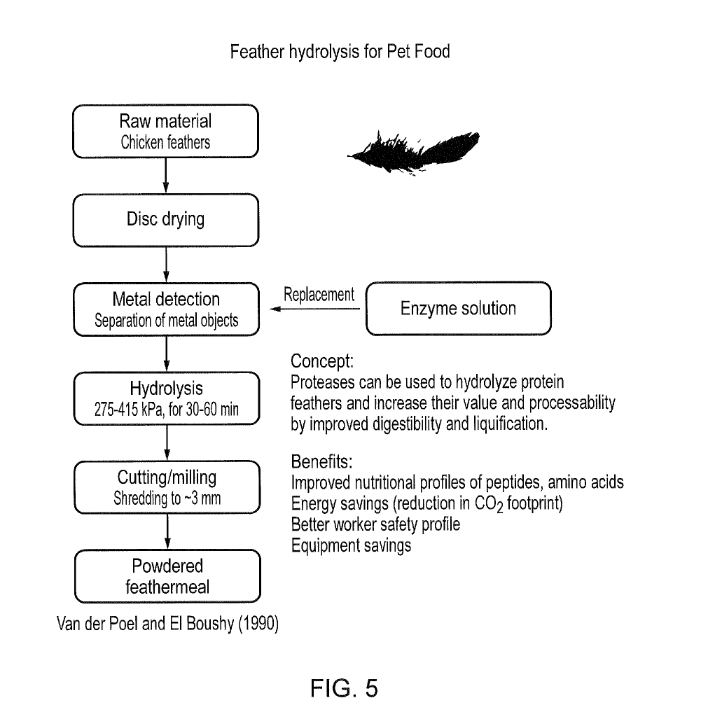

FIG. 5 shows an embodiment for the use of enzymatic feather hydrolysis.

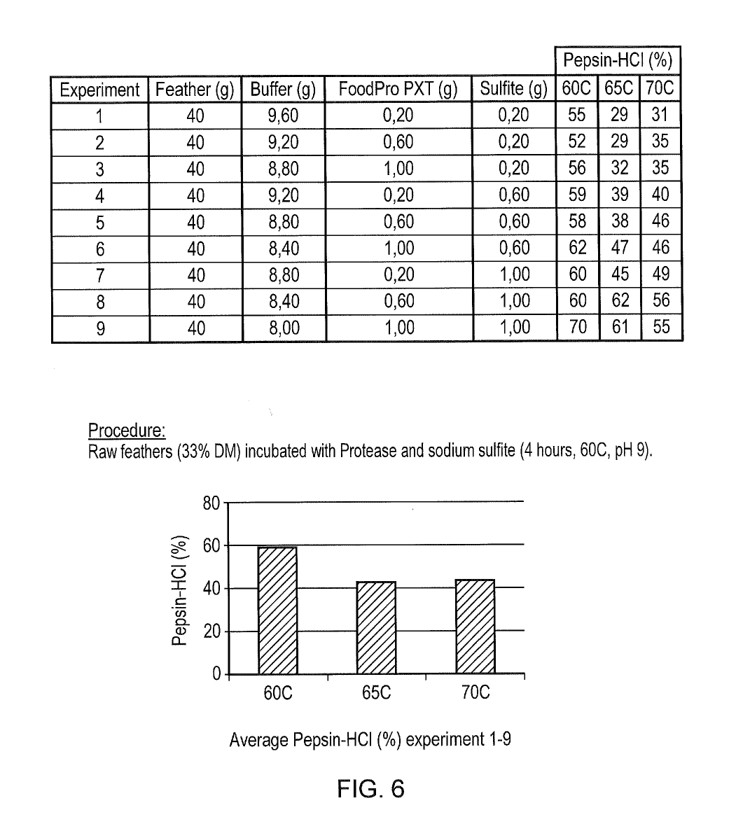

FIG. 6 shows results for feather hydrolysis replacement.

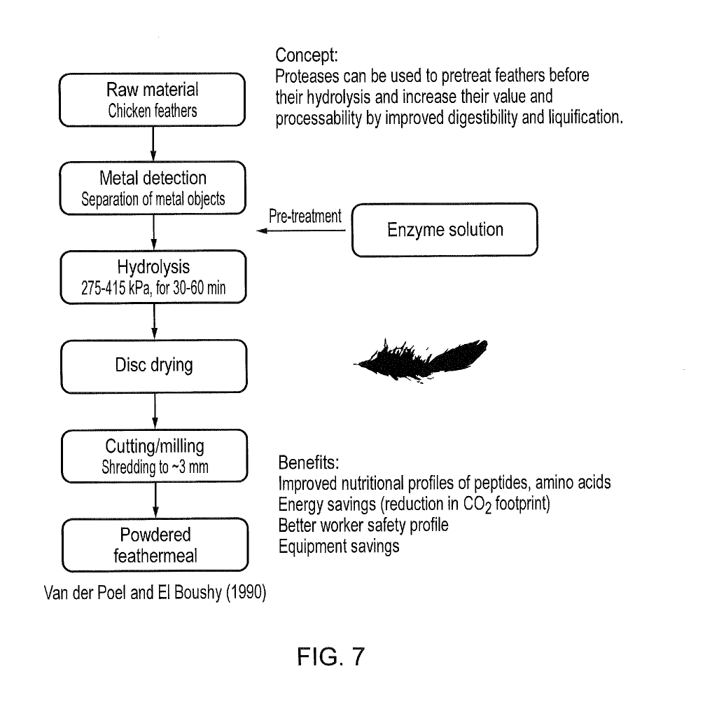

FIG. 7 shows an embodiment for the use of enzymatic feather hydrolysis.

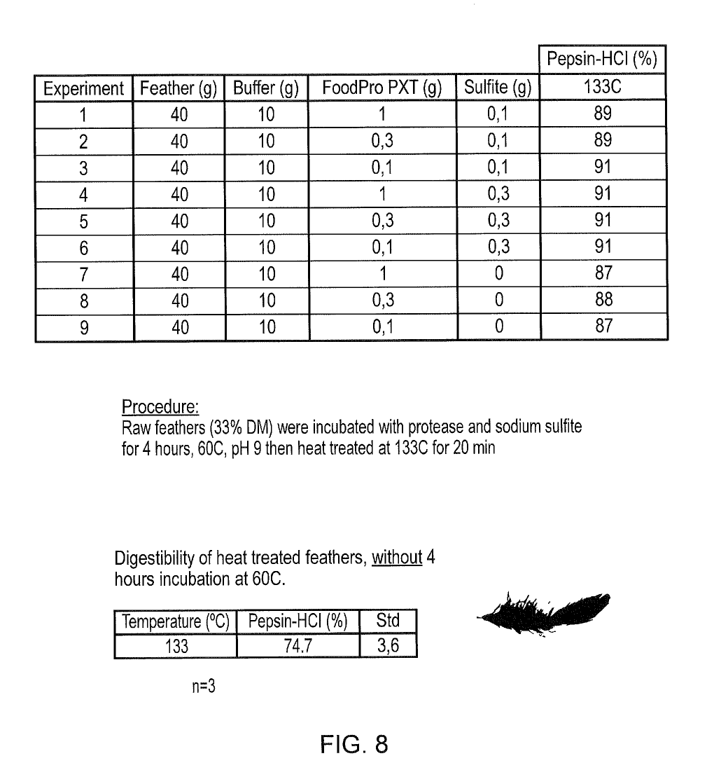

FIG. 8 shows results for feather hydrolysis with protease pretreatment.

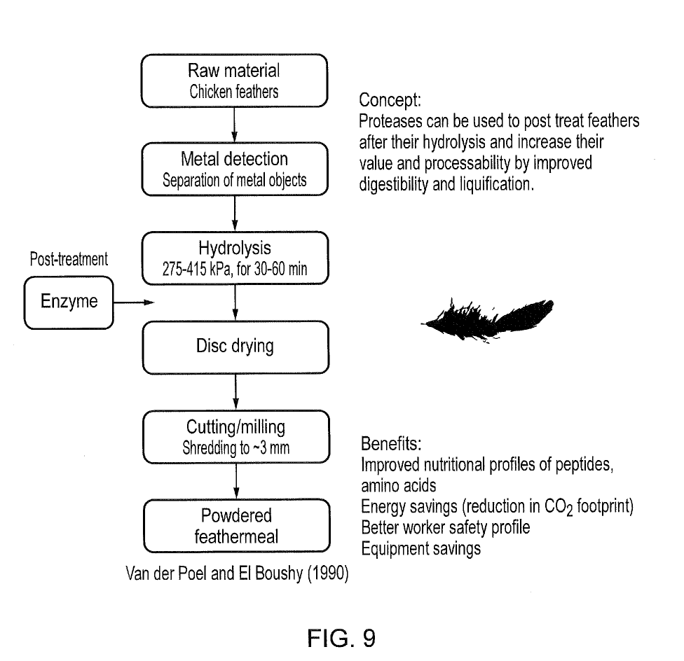

FIG. 9 shows an embodiment for the use of enzymatic feather hydrolysis.

A. Enzymatic Hydrolysis Post Heating Treatment

MATERIALS: Acetic acid (Sigma; 33209), Bis-Tris (Sigma; B9754), CHES (Sigma; C2885), HEPES (Sigma; H3375), Sodium sulfite (Sigma; 71988), Hexane (Sigma; 296090), Meat grinder with 4.5 mm disc (Assistent), FoodPro® PXT (DuPont), Pepsin BD Difco™ 1:10.000 units (BD, Franklin Lakes, N.J., USA), HCl (Sigma; 320331), Glassfiber filter (Whatman 1.5 μM pore size; Springfield Mill, Kent, UK).

Chicken feathers were ground on a standard meat grinder with a 4.5 mm disc size. 40 g ground chicken feathers (33% w/w dry matter) were weighed into metal beakers with 40 g steel balls and mixed with 10 g buffer (0.5 M Acetic acid/Bis-Tris/Ches/Hepes pH 9). Beakers were heated 20 min at 123° C. Beakers were cooled to 60° C. and FoodPro® PXT and sodium sulfite added as listed in table 1. Samples incubated in a Laudry-Ometer® machine (SDL-Atlas) with rotary mixing at 60° C. for 4 hours. Samples were heated 10 min at 95° C. to inactive enzymes. Samples were transferred to 50 ml tubes and centrifuged (4.000 RPM, 10 min, 25° C.). The supernatant was removed and 15 ml hexane added to the pellet to defat. Samples were left to mix on a rotary wheel for 30 min. Samples were centrifuged at (4.000 RPM, 10 min, 25° C.), and supernatant discarded. Pellets were dried in a freeze dryer until weights were constant. The dried pellet was weighed (W2). 1 g of freeze dried material was weighted into 250 ml blue cap bottles. 150 ml 0.075 M HCl, Pepsin (2 mg/ml) was added and incubated at 45° C. for 16 hours with magnetic stirring. Solutions were filtered on a whatman filter (1.5 um particle retention). The filter was dried in an oven at 80° C. until a constant weight and weighed (W3).

Soluble dry matter yield (%)=((W1−W2)/W1)×100%

Digestible residue (%)=(100−W2/W1*100)+(100−(100−W2/W1*100))*(1−W3)

W1: Amount of dry matter at start of experiment

W2: Amount of insoluble dry matter

W3: Amount of dry matter on filter after pepsin digestion

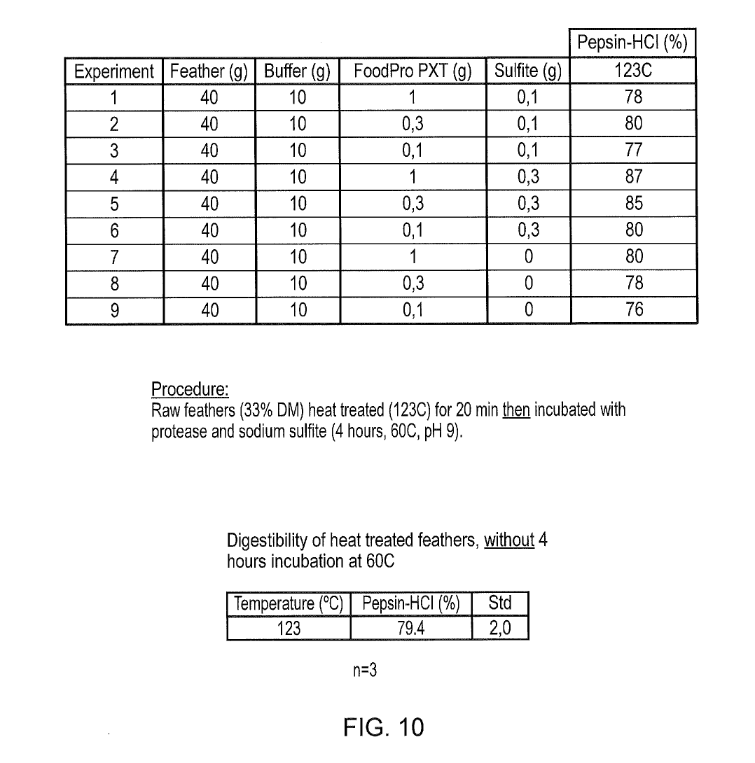

FIG. 10 and the table below show results for feather hydrolysis for protease post treatment.

TABLE 1

| | | | | | Soluble | |
|---|---|---|---|---|---|---|
| | | | FoodPro | Sodium | dry matter | Digestible |
| Exper- | Feather | Buffer | PXT | Sulfite | yield | residue |
| iment | (g) | (g) | (g) | (g) | (%) | (%) |
| 1 | 40 | 10 | 1 | 0.1 | 45.5 | 78.3 |
| 2 | 40 | 10 | 0.3 | 0.1 | 38.6 | 79.8 |
| 3 | 40 | 10 | 0.1 | 0.1 | 27.7 | 77.5 |
| 4 | 40 | 10 | 1 | 0.3 | 65.2 | 86.7 |
| 5 | 40 | 10 | 0.3 | 0.3 | 59.1 | 85.4 |
| 6 | 40 | 10 | 0.1 | 0.3 | 47.7 | 80.4 |
| 7 | 40 | 10 | 1 | 0 | 48.3 | 79.9 |
| 8 | 40 | 10 | 0.3 | 0 | 34.4 | 77.8 |
| 9 | 40 | 10 | 0.1 | 0 | 26.2 | 75.9 |
| 10 | 40 | 10 | 0 | 0 | −0.6 | 75.2 |

Heat treated chicken feathers lost its macromolecular structure upon treatment with FoodPro® PXT and the combination of FoodPro® PXT and sodium sulfite. Surprisingly, FoodPro® PXT was able to solubilize high amount of dry matter in the feather, indicating that the flavor and likely the palatability of the material had increased. Also, the digestibility of the chicken feathers as measured by the digestible residue increased compared to the reference both upon treatment with FoodPro® PXT and the combination of FoodPro® PXT and sodium sulfite.

B. Enzymatic Hydrolysis Post Heating Treatment

MATERIALS: Acetic acid (Sigma; 33209), Bis-Tris (Sigma; B9754), CHES (Sigma; C2885), HEPES (Sigma; H3375), Sodium sulfite (Sigma; 71988), Hexane (Sigma; 296090), Meat grinder with 4.5 mm disc (Assistent), FoodPro® PXT (DuPont), Pepsin BD Difco™ 1:10.000 units (BD, Franklin Lakes, N.J., USA), HCl (Sigma; 320331), Glassfiber filter (Whatman 1.5 μM pore size; Springfield Mill, Kent, UK).

Chicken feathers were ground on a meat grinder with a 4.5 mm disc size. 40 g ground chicken feathers (33 w/w % dry matter) were weighed into metal beakers with 40 g steel balls and mixed with buffer (0.5 M Acetic acid/Bis-Tris/Ches/Hepes pH 9), FoodPro PXT, and sodium sulfite according to table 2. Samples incubated in a Laudry-Ometer® machine (SDL-Atlas) with rotary mixing at 60° C. for 4 hours. Samples were heated 20 min at 133° C. in an autoclave. 125 ml water was added to each sample. Material was transferred and centrifuged in 50 ml tubes (4.000 rpm, 10 min, 25° C.). The supernatant was removed and 15 ml hexane added to the pellet to defat. Samples were left to mix on a rotary wheel for 30 min. Samples centrifuged (4.000 rpm, 10 min, 25° C.), and supernatant discarded. Pellets were dried in a freeze dryer until weights were constant. The dried pellet was weighed (W2). 1 g of freeze dried material was weighted into 250 ml blue cap bottles. 150 ml 0.075 M HCl, Pepsin (2 mg/ml) was added and incubated at 45° C. for 16 hours with magnetic stirring. Solutions were filtered on a whatman filter (1.5 um particle retention). The filter was dried in an oven at 80° C. until a constant weight and weighed (W3).

Soluble dry matter yield (%)=((W1−W2)/W1)×100%

Digestible residue (%)=(100−W2/W1*100)+(100−(100−W2/W1*100))*(1−W3)

W1: Amount of dry matter at start of experiment

W2: Amount of insoluble dry matter

W3: Amount of dry matter on filter after pepsin digestion

TABLE 2

| | | | | | Soluble | |
|---|---|---|---|---|---|---|
| | | | FoodPro | Sodium | dry matter | Digestible |
| Exper- | Feather | Buffer | PXT | Sulfite | yield | residue |
| iment | (g) | (g) | (g) | (g) | (%) | (%) |
| 1 | 40 | 10 | 1 | 0.1 | 46.1 | 89.4 |
| 2 | 40 | 10 | 0.3 | 0.1 | 41.7 | 88.6 |
| 3 | 40 | 10 | 0.1 | 0.1 | 47.7 | 91.4 |
| 4 | 40 | 10 | 1 | 0.3 | 48.4 | 90.7 |
| 5 | 40 | 10 | 0.3 | 0.3 | 49.2 | 91.3 |
| 6 | 40 | 10 | 0.1 | 0.3 | 44.3 | 91.2 |
| 7 | 40 | 10 | 1 | 0 | 38.7 | 86.8 |
| 8 | 40 | 10 | 0.3 | 0 | 38.2 | 88.3 |
| 9 | 40 | 10 | 0.1 | 0 | 38.2 | 86.9 |
| 10 | 40 | 10 | 0 | 0 | 24.9 | 85.9 |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of production of keratin hydrolysate comprising the steps of:
a) processing a keratin-containing material in a size-reduction process to produce a size-reduced keratin material, wherein said size-reduced keratin material comprises an average particle size of 5 mm or less,
b) admixing said size-reduced keratin material with one or more proteases, and
c) hydrolyzing said keratin material by heat hydrolysis, wherein step b) and c) can occur in any order.

2. The method of claim 2, wherein said heat hydrolysis is steam hydrolysis.

3. The method of claim 2, wherein the steam has a temperature of from about 120° C. to about 140° C.

4. The method of any preceding claim, wherein said heat hydrolysis occurs prior to the enzyme hydrolysis step.

5. The method of any preceding claim, wherein the steam or heat hydrolysis occurs simultaneously with the size-reducing step prior to the enzyme hydrolysis step.

6. The method of claim 1 or 2, wherein said heat hydrolysis occurs after to the enzyme hydrolysis step.

7. The method of any preceding claim, wherein no further chemical hydrolysis of the keratin material is performed.

8. The method of any preceding claim, wherein the protease is a subtilisin, a serine protease, a metalloprotease, an acid protease, or a neutral protease.

9. The method of claim 8, wherein said protease has at least 80, 81, 82, 83, 84, 85, 86, 87, 88, 89, 90, 91, 92, 93, 94, 95, 96, 97, 98 or 99% identity with the amino acid SEQ ID No.1.

10. The method of claim 9, wherein said protease has at least 90% identity with the amino acid SEQ ID No.1.

11. The method of claim 10, wherein said protease has at least 95% identity with the amino acid SEQ ID No.1.

12. The method of claim 8, wherein said protease has at least 80, 81, 82, 83, 84, 85, 86, 87, 88, 89, 90, 91, 92, 93, 94, 95, 96, 97, 98 or 99% identity with the amino acid SEQ ID No.2.

13. The method of claim 12, wherein said protease has at least 90% identity with the amino acid SEQ ID No.2.

14. The method of claim 13, wherein said protease has at least 95% identity with the amino acid SEQ ID No.2.

15. The method of any preceding claim, wherein the heat hydrolysis step is carried out at +/− about 1 pH of the optimal pH of the protease.

16. The method of any preceding claim, wherein the heat hydrolysis step is carried out at +/− about 0.5 pH of the optimal pH of the protease.

17. The method of any preceding claim, wherein the optimal pH of the protease is 6-10.

18. The method of any preceding claim, wherein the keratin material is feathers.

19. The method of any preceding claim, wherein said keratin material is reduced to an average particle size of about 4.5 mm or less, or less than 4 mm, or less than 3 mm, or less than 2 mm, or less than 1 mm.

20. The method of any preceding claim wherein said protease is in a liquid composition.

21. The method of claim 20 wherein said liquid composition comprise protease activity of 2500-3600 U/g.

22. The method of any preceding claim wherein the protease is present in an amount of 0.3 g-9 g per Kg of keratin-containing proteinaceous material.

23. The method of claim 22, wherein said protease comprise protease activity of 2500-3600 U/g.

24. The method of any preceding claim, wherein the method is carried out in an oxygen controlled environment.

25. The method of any preceding claim, wherein the method is carried out as a batch, fed-batch or continuous process.

26. A keratin hydrolysate comprising at least about 50% of peptides with N-terminal Leucine, Isoleucine, Valine, Alanine, Tyrosine, Phenylalanine or Lysine.

27. The keratin hydrolysate of claim 26 comprising at least about 70% of peptides with N-terminal Leucine, Isoleucine, Valine, Alanine, Tyrosine, Phenylalanine or Lysine.

28. The keratin hydrolysate of claim 26 comprising at least about 80% of peptides with N-terminal Leucine, Isoleucine, Valine, Alanine, Tyrosine, Phenylalanine or Lysine.

29. The keratin hydrolysate of claim 26 comprising at least about 90% of peptides with N-terminal Leucine, Isoleucine, Valine, Alanine, Tyrosine, Phenylalanine or Lysine.

30. The keratin hydrolysate of claims 26-29, wherein 80% of the keratin hydrolysates comprise peptides having a molecular weight in the range of from about 0.1 kDa to about 5 kDa.

31. The keratin hydrolysate of claims 26-30, wherein 80% of the keratin hydrolysates comprise peptides having a molecular weight in the range of from about 0.1 kDa to about 1.0 kDa.

32. The keratin hydrolysate of claims 26-31, wherein 80% of the keratin hydrolysates comprise peptides having a molecular weight in the range of from about 0.1 kDa to about 0.8 kDa.

33. The keratin hydrolysate of claims 26-32, wherein the keratin hydrolysates have 50% less undesirable amino acids that result from chemical hydrolysis when compared to a comparable keratin hydrolysate produced using chemical hydrolysis.


SEQUENCE LISTING (6 sequences)

SEQ ID NO: 1 - protein, 269 aa, Artificial Sequence
  Protease amino acid sequence
AQSVPWGISRVQAPAAHNRGLTGSGVKVAVLDTGISTHPDLNIRGGASFVPGEPSTQDGNGHGTHVAGTIAALDNSIGVLGVAPSAELYAVKVLGASGSGAISSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 2 - protein, 274 aa, Artificial Sequence
  Protease amino acid sequence
AQTVPYGIPLIKADKVQAQGFKGANVKVAVLDTGIQASHPDLNVVGGASFVAGEAYNTDGNGHGTHVAGTVAALDNTTGVLGVAPSVSLYAVKVLNSSGSGSYSGIVSGIEWATTNGMDVINMSLGGASGSTAMKQAVDNAYARGVVVVAAAGNSGSSGNTNTIGYPAKYDSVIAVGAVDSNSNRASFSSVGAELEVMAPGAGVYSTYPTNTYATLNGTSMASPHVAGAAALILSKHPNLSASQVRNRLSSTATYLGSSFYYGKGLINVEAAAQ

SEQ ID NO: 3 - protein, 98 aa, Gallus gallus
MSCFDLCRPCGPTPLANSCNEPCVRQCQDSRVVIQPSPVVVTLPGPILSSFPQNTAAGSSTSAAVGSILSEEGVPISSGGFGISGLGSRFSGRRCLPC

SEQ ID NO: 4 - protein, 98 aa, Gallus gallus
MSCYDLCRPCGPTPLANSCNEPCVRQCQDSRVVIQPSPVVVTLPGPILSSFPQNTAVGSSTSAAVGSILSEEGVPISCGGFGISGLGSRFSGRRCLPC

SEQ ID NO: 5 - protein, 98 aa, Gallus gallus
MSCFDLCRPCGPTPLANSCNEACVRQCQDSRVVIQPSPVVVTLPGPILSSFPQNTLVGSSTSAAVGSILSEEGVPISSGGFGISGLGSRFSGRRCLPC

SEQ ID NO: 6 - protein, 98 aa, Gallus gallus
MSCYDLCRPSAPTPLANSCNEPCVRQCQDSRVVIQPSPVVVTLPGPILSSFPQNTAVGSSTSAAVGSILSEEGVPISSGGFGISGLGSRFSSRRCLPY